(19) United States
(12) Patent Application Publication
(10) Pub. No.: US 2026/0007621 A1
(43) Pub. Date: Jan. 8, 2026

(54) Pharmaceutical Combinations for Treatment

(71) Applicant: MFS PHARMACEUTICALS LIMITED, Lower Hutt (NZ)

(72) Inventor: Faran Shahzad, Sydney (AU)

(73) Assignee: MFS Pharmaceuticals Ltd., Sydney (AU)

(21) Appl. No.: 19/124,744

(22) PCT Filed: Oct. 30, 2023
(86) PCT No.: PCT/NZ2023/050116

§ 371 Date: Apr. 25, 2025

(30) Foreign Application Priority Data
Oct. 28, 2022 (AU) 2022259844

(51) Int. Cl. A61K 31/167; A61K 31/09; A61K 31/137; A61K 31/192; A61K 31/445; A61K 31/454; A61K 31/4545; A61K 31/485; A61K 31/495; A61P 1/08; A61P 11/02; A61P 11/10; A61P 11/14; A61P 25/06

(52) U.S. Cl. CPC A61K 31/167; A61K 31/09; A61K 31/137; A61K 31/192; A61K 31/445; A61K 31/454; A61K 31/4545; A61K 31/485; A61K 31/495; A61P 1/08; A61P 11/02; A61P 11/10; A61P 11/14; A61P 25/06

(57) ABSTRACT

This invention relates to the use of ingredients comprising paracetamol, ibuprofen, and any one or both of antihistamine and guaifenesin (or a pharmaceutically acceptable salt, metabolite, or enantiomer of any mentioned aforesaid) in the production of a medication for the treatment of any one or more of mucosal congestion, coughing, fever, sinus secretions, muscle ache, malaise, cold, flu and headache in a human.

### CROSS-REFERENCE TO RELATED APPLICATIONS

The present application is the national phase of PCT App. No. PCT/NZ2023/050116, filed Oct. 30, 2023, which is incorporated herein by reference.

### BACKGROUND

### Field of the Invention

This invention relates to pharmaceutical compositions comprising guaifenesin (GFN), acetaminophen (APAP), ibuprofen (IBF), and antihistamine(s) (e.g., cetirizine, bilastine, loratadine, fexofenadine etc) and a method of administering the compositions. The invention particularly, though not exclusively, relates to the treatment of respiratory illnesses.

### Background

Illnesses, particularly respiratory illness, caused by infections can lead to a huge number of unpleasant symptoms out of which the main symptoms are mucosal congestion, coughing, fever, sinus secretions, muscle ache, malaise, cold, flu and headache.

The aim of the preferred forms of the invention is to go at least some way towards providing pharmaceutical combinations comprising any of GFN, APAP, IBF, and antihistamine(s) (e.g., cetirizine, bilastine, loratadine, fexofenadine etc) for relieving at least some of the above symptoms for at least some people.

Popular brand names (e.g., Duraflu, Robitussin Cold Cough and Flu, Theraflu Max-D Severe Cold & Flu etc) include ingredients such as dextromethorphan (DM), GFN, Phenylephrine (PE) and APAP.

Please refer to the table below for known dosages.

TABLE 1
Known Dosages

| | | Maximum |
|---|---|---|
| Active Ingredients | Dosage | dosage |
| 325 mg APAP/10 mg | 2 tablets | 5 doses |
| DM/200 mg GFN/5 mg PE | every 4 hours | (10 tablets) |
| per tablet | | per 24 hours |
| 325 mg APAP/10 mg | 2 softgels | 6 doses |
| DM/200 mg GFN/5 mg PE | every 4 hours | (12 softgels) |
| per softgel | | per 24 hours |
| 325 mg APAP/10 mg | 20 mL | 6 doses |
| DM/200 mg GFN/5 mg PE | every 4 hours | per 24 hours |
| per 10 mL liquid | | |
| 325 mg APAP/10 mg | 30 mL | 4 doses |
| DM/200 mg GFN/5 mg PE | every 4 hours | per 24 hours |
| per 15 mL liquid | | |
| 650 mg APAP/20 mg | 20 mL | 6 doses |
| DM/400 mg GFN/10 mg PE | every 4 hours | per 24 hours |
| per 20 mL liquid | | |
| 650 mg APAP/20 mg | 30 mL | 4 doses |
| DM/400 mg GFN/10 mg PE | every 4 hours | per 24 hours |
| per 30 mL liquid | | |

DM+GFN+PE+APAP is a popular combination in over the counter (OTC) cough medicines. DM is a cough suppressant and works by activating the sigma opioid receptors in the central nervous system which leads to the cough reflex being supressed. GFN is an expectorant and works by loosening mucus in the airway which in turn makes the coughing more productive in expelling excess mucus. PE is a decongestant and it works by reducing the size of blood vessels in the nose and sinus to allow easier breathing. APAP is a painkiller and is used to treat fever and mild to moderate pain.

The full daily dose limit for DM, GFN, PE and APAP is 120 mg, 2,400 mg, 60 mg and 4,000 mg respectively. The full daily dose limit allowed for DM can lead to mild hallucinations. Vehicle drivers are warned against using DM during driving.

The current versions of medicine (e.g., cough syrups) comprising DM, GFN, PE and APAP are either not effective, are prone to drug abuse, have multiple negative side effects, are discouraged from being supplied or are unavailable to those who are in dire need. The problems are caused due to the way the individual ingredients, in particular DM, GFN and PE, are used.

### Problem with PE

Before PE was used in cough medicines, pharmaceutical companies used pseudoephedrine (PSE). However, there was a back lash against the use of PSE as it was being abused to produce methamphetamine. One common method used by abusers was to place OTC tablets containing PSE in a solvent to separate PSE and then produce methamphetamine using internet recipes and common household products. Since 2004, many governments have banned the use of PSE in OTC medicines.

The pharmaceutical industry now uses PE as an alternative to PSE. However, studies have shown that PE is not as effective as PSE (Hatton RC et al, The Annals of Pharmacotherapy, 2007 March, Vo. 41).

### Decreasing PSE Abuse and Increasing Relief Provided by PE

$$PSE + Iysine$$

The inventor has suggested using a version of PSE such that it cannot be abused. This is done by attaching a peptide (e.g., lysine etc), through a covalent bond using a hydroxyl group, with PSE. Bonding PSE with lysine protects PSE from in-vitro extraction and therefore preventing abuse. That said, the human body can extract PSE by using gut enzymes to cleave off lysine. Canadian Patent No. 2,540,678 teaches protecting singe ingredients of a medicine by using lysine. However, the above patent does not teaches protecting pharmaceutical combinations mentioned in this specification. Lysine is also called a prodrug. A prodrug improves the stability, absorption, solubility, duration, specificity and decreases toxicity of the medication. Therefore, PSE+lysine also improves the overall medication apart from protecting PSE from being abused.

$$( APAP and / or IBF ) + Antihistamine + PE$$

As an alternative, the inventor also suggests using PE with APAP, which increases relief provided by PE. It is shown in AU2013211546 that APAP enhances the effect of PE such that when 6.25 mg PE is administered with 500 mg APAP then the effect is similar to a dosage of 10 mg PE (Reference: page 8, line 19-23 of AU2013211546 specification). Therefore, it is known that relief provided by PE will be increased when combined with APAP. While AU2013211546 teaches using APAP+PE it does not mention combining APAP+PE alongside other active ingredients (e.g. antihistamine etc) stated in this specification.

It is shown in NZ541960 that antihistamine Loratadine and PE have a synergic effect on each other and leads to increase in overall effectiveness when combined together (Reference: page 9, lines 5-10 of NZ541960 specification). That said, the combination of Loratadine+PE was still found to be less efficient than PSE (Reference: page 8, lines 25-27 of NZ541960 specification).

The inventor has found that PE when combined with APAP and/or IBF significantly increased therapeutic effect of antihistamines (e.g., e.g., cetirizine, bilastine loratadine, fexofenadine etc) in comparison to when PE and antihistamines were combined alone. Furthermore, the inventor found that synergic enhancement and efficiency increased when PE was combined with antihistamine, APAP and/or IBF such that the overall combination had therapeutic effect greater than that of DM+GFN combination. This is surprising because DM is a cough suppressant and GFN is an expectorant while the other ingredients (e.g., cetirizine, bilastine, loratadine, fexofenadine, APAP, PE and IBF) are either a decongestant, antihistamine(s), or painkillers and not expected to fully replace a cough suppressant and an expectorant.

The benefit of the PE+antihistamine+APAP and/or IBF combination is that less amount of PE and antihistamine has to be used to give the therapeutic effect of a higher amount, the combination omits or decreases the use of DM and GFN, the combination will decrease drug abuse, vendors are more likely to stock it as OTC medication due to decreased negative effects and government restrictions and therefore more available to those in dire need of the medication.

### Problem with DM and GFN

High amounts of DM and GFN can lead to symptoms such as stomach pain, diarrhoea, acid reflex etc. A lot of places have banned OTC medicines comprising DM due to it being abused. DM is amongst the most abused OTC medication due to its hallucinogen effects. DM addiction is commonly found within teenagers. Deaths due to its abuse are found all over the world. One study by Humera Shafi et al reports 50 deaths in Pakistan (Reference: Humera Shafi, Muhammad Imran, Hafiz Faisal Usman, Muhammad Sarwar, Muhammad Ashraf Tahir, Rabia Naveed, Muhammad Zar Ashiq, Ammar M. Tahir, Deaths due to abuse of dextromethorphan sold over-the-counter in Pakistan, Egyptian Journal of Forensic Sciences, Volume 6, Issue 3, 2016, Pages 280-283).

Due to the popular DM+GFN combination in medicines, abusers of DM also end up abusing GFN. Nowadays, experienced DM abusers are extracting DM from medications to avoid coingestant toxicity from other active ingredients.

Due to DM's and GFN's gastric side effects, it is not suitable for people with gastric problems. Furthermore, medicines with high dosage of DM+GFN are banned, as OTC medicines, in lots of places and even in places allowed, the vendors are discouraged from selling it. This has negatively affected those people who are in genuine and urgent need of treatment.

### Reducing Amount of DM and GFN, and Increasing Medication Efficiency

$$APAP + IBF + GFN$$

It is shown in EP 0529898A1 that a painkiller APAP enhances the effect of DM however it was not known, as found by the inventor, that a painkiller combination of IBF+APAP enhances the effect of GFN and provides relief such that DM is no longer needed or will have to be used in decreased amounts. Therefore, with the addition of IBF+APAP, a lower dosage of GFN gives an effect of a higher dosage. By omitting or decreasing DM from the medicines containing the DM+GFN combination, DM abusers will not be able to abuse DM, vendors will be more likely to stock GFN containing medicines, the active ingredient GFN will be more efficiently used, and a user will experience results one gets from a higher dosage but without the negative side effects experienced due to the ingestion of large amounts of GFN.

It is suggested that low doses flatten the differences between the peak and trough drug concentrations in the blood plasma which in turn decreases the negative side effects and is beneficial for a stable homeostatic system. An increased, evenly spaced, frequency of low dosing maintains a low constant drug (e.g IBF+APAP+GFN) concentration in the plasma (a result which mimics continuous dosing) and maximises the advantages gained from those drugs.

$$APAP + IBF + Antihistamines$$

It is shown in AU2005260243B2 and AU2013211546B1 that APAP enhances the effect of IBF and PE; however, surprisingly, as found by the inventor, that APAP and IBF, when used together, have a significant enhancing effect on antihistamines. In particular second-generation OTC antihistamines (e.g., cetirizine, bilastine, loratadine etc) and third-generation OTC antihistamines (e.g.,fexofenadine etc). Fexofenadine is safe enough such that it has been approved for treating chronic urticaria in children older than 6 months and for treating seasonal allergic rhinitis in children older than two years. The second and third generation antihistamines work by inhibiting H1 receptors and cause less drowsiness because they have difficulty in passing the blood brain barrier and are taken in lower doses than its first-generation counterparts. The full daily dose limit for IBF, cetirizine, bilastine, loratadine and Fexofenadine is 3,200 mg, 10 mg, 20 mg, 10 mg, and 180 mg respectively.

While antihistamines primarily treat allergies, they can also be used to treat cold and cough symptoms such as sneezing, itchiness, watery eyes, runny noise etc because there is a big overlap between the cold and allergy symptoms.

The inventor has further found that antihistamines (e.g., cetirizine, bilastine loratadine, fexofenadine etc) when taken with APAP and IBF decrease or omit the need of using DM and GFN. This is surprising because DM is a cough suppressant and GFN is an expectorant while the rest of the ingredients (e.g., cetirizine, bilastine, loratadine, fexofenadine, APAP and IBF) are either antihistamine(s) or painkillers and not expected to fully replace a cough suppressant and an expectorant. Normally in a medication only one antihistamine is used. It is suggested that using a combination of antihistamines (e.g., cetirizine+loratadine, loratadine+fexofenadine etc) along with APAP and IBF leads to a better relief. It is found that APAP and IBF complement each other and have an enhancing effect on the relief provided by the antihistamines leading to DM and GFN being decreased or omitted from medicine. Similarly, it is suggested when two or more antihistamines are used together, for example cetirizine +loratadine, then they also compliment each other and therefore result in better relief in comparison to one antihistamine being used.

### Definitions

The term “comprising” or “has,” as used in this document in relation to a series of features, means they are present as a minimum combination but does not rule out the option of there being additional features. The same applies to related terms such as “comprises” or “having.”

References in this document to a “day” mean a 24-hour period.

### SUMMARY OF THE INVENTION

### First Aspect—Use in Manufacture

Use of ingredients comprising paracetamol, ibuprofen, and any one or both of antihistamine and guaifenesin (or a pharmaceutically acceptable salt, metabolite, or enantiomer of any mentioned aforesaid) in the production of a medication for the treatment of any one or more of mucosal congestion, coughing, fever, sinus secretions, muscle ache, malaise, cold, flu and headache in a human.

Optionally the antihistamine comprises any one or more of cetirizine, bilastine, loratadine, and fexofenadine (or a pharmaceutically acceptable salt, metabolite, or enantiomer of any mentioned aforesaid).

Optionally the medication comprises any one or more of phenylephrine, pseudoephedrine, and dextromethorphan (or a pharmaceutically acceptable salt, metabolite, or enantiomer of any mentioned aforesaid).

Optionally one or more of the ingredients paracetamol, ibuprofen, guaifenesin, cetirizine, bilastine, loratadine, fexofenadine, phenylephrine, pseudoephedrine and dextromethorphan are combined in a dosage unit.

Optionally one or more of the ingredients paracetamol, ibuprofen, guaifenesin, cetirizine, bilastine, loratadine, fexofenadine, phenylephrine, pseudoephedrine and dextromethorphan are in separate dosage units.

Optionally the medication is for administering or taking in a 24-hour period at least one of the following ingredients in the amounts indicated for that ingredient:
a. paracetamol between 1-4,000 mg;
b. ibuprofen between 1-3,200 mg;
c. guaifenesin between 1-2,400 mg;
d. cetirizine between 1-10 mg;
e. bilastine between 1-100 mg;
f. loratadine between 1-10 mg;
g. fexofenadine between 1-180 mg;
h. phenylephrine between 1-60 mg;
i. pseudoephedrine between 1-240 mg; and
j. dextromethorphan between 0-120 mg.

Optionally the medication is for administering or taking in the 24-hour period at least one of the following ingredients in the amounts indicated for that ingredient:
a. paracetamol between 350-4,000 mg;
b. ibuprofen between 100-600 mg;
c. guaifenesin between 300-2,400 mg;
d. cetirizine between 1-10 mg;
e. bilastine between 5-100 mg;
f. loratadine between 1-10 mg;
g. fexofenadine between 30-180 mg;
h. phenylephrine between 1-40 mg;
i. pseudoephedrine between 60-240 mg; and
j. dextromethorphan between 0-120 mg.

Optionally the medication is for administering or taking in 1-10 dosage units during the 24-hour period.

Optionally the medication is for administering or taking in a 12-hour period at least one of the following ingredients in the amounts indicated for that ingredient:
a. paracetamol between 1-2,000 mg;
b. ibuprofen between 1-600 mg;
c. guaifenesin between 1-1,200 mg;
d. cetirizine between 1-5 mg;
e. bilastine between 1-80 mg;
f. loratadine between 1-5 mg;
g. fexofenadine between 1-90 mg;
h. phenylephrine between 1-30 mg;
i. pseudoephedrine between 1-120 mg; and
j. dextromethorphan between 0-80 mg.

Optionally the medication is for administering or taking in 1-5 dosage units during the 12-hour period.

Optionally the medication is for administering or taking at least one of the following ingredients in the amounts indicated for that ingredient:
a. paracetamol between 300-600 mg;
b. ibuprofen between 100-300 mg;
c. guaifenesin between 80-380 mg;
d. cetirizine between 1-10 mg;
e. bilastine between 1-20 mg;
f. loratadine between 1-10 mg;
g. fexofenadine between 1-200 mg;
h. phenylephrine between 1-20 mg;
i. pseudoephedrine between 5-70 mg; and
j. dextromethorphan between 0-25 mg.

Optionally the medication is for administering or taking at least one of the following ingredients in the amounts indicated for that ingredient:
a. paracetamol between 275-575 mg;
b. ibuprofen between 125-250 mg;
c. guaifenesin between 100-300 mg;
d. cetirizine between 1.5-10 mg;
e. bilastine between 1.5-20 mg;
f. loratadine between 1.5-10 mg;
g. fexofenadine between 20-200 mg;
h. phenylephrine between 1-10 mg;
i. pseudoephedrine between 15-55 mg; and
j. dextromethorphan between 0-20 mg.

Optionally the medication is for administering or taking at least one of the following ingredients in the amounts indicated for that ingredient:
a. paracetamol between 425-500 mg;
b. ibuprofen between 125-200 mg;
c. guaifenesin between 100-200 mg;
d. cetirizine between 2-10 mg;
e. bilastine between 4-20 mg;
f. loratadine between 2-10 mg;
g. fexofenadine between 40-180 mg;
h. phenylephrine between 2.5-8 mg;
i. pseudoephedrine between 20-50 mg; and
j. dextromethorphan between 0-10 mg.

Optionally the medication is for administering or taking at least one of the following ingredients in the amounts indicated for that ingredient:
a. paracetamol 500 mg;
b. ibuprofen 150 mg;
c. guaifenesin 150 mg;
d. cetirizine 2.5 mg;
e. bilastine 5 mg;
f. loratadine 2.5 mg;
g. fexofenadine 30 mg;
h. phenylephrine 5 mg;
i. pseudoephedrine 30 mg; and
j. dextromethorphan between 0-5 mg.

Optionally the medication further comprises a prodrug.

Optionally the prodrug is a peptide covalently linked to pseudoephedrine.

Optionally the peptide is attached by a hydroxyl group to pseudoephedrine.

Optionally the prodrug is lysine.

Optionally the paracetamol, the ibuprofen and any one or both of the antihistamine and the guaifenesin (or a pharmaceutically acceptable salt, metabolite, or enantiomer of any mentioned aforesaid) are for administering or taking at the same dosage event, on at least some occasions during the same day.

Optionally the paracetamol and the ibuprofen and any one or both of the antihistamine and the guaifenesin (or a pharmaceutically acceptable salt, metabolite, or enantiomer of any mentioned aforesaid) are for administering or taking at different dosage events, on at least some occasions during the same day.

Optionally the medication is such that the paracetamol and the ibuprofen and any one or both of the antihistamine and the guaifenesin (or a pharmaceutically acceptable salt, metabolite, or enantiomer of any mentioned aforesaid) are for administering or taking in doses comprising of one or more of the following forms: a) tablets, b) capsule, c) aerosol and d) liquid.

Optionally the medication is such that the paracetamol, the ibuprofen and the antihistamine (or a pharmaceutically acceptable salt, metabolite, or enantiomer of any mentioned aforesaid) are for administering or taking in doses comprising one or both of the following forms a) tablets and b) capsules.

Optionally the medication is such that the guaifenesin (or a pharmaceutically acceptable salt, metabolite, or enantiomer thereof) is for administering in doses comprising one or both of the following forms a) liquid and b) aerosol form.

Optionally the medication is contained in a kit with instructions on how to take the medication.

Optionally the kit has two sections such that section 1 contains a combination of any one or more of phenylephrine, pseudoephedrine and dextromethorphan while section 2 does not have the combination of phenylephrine, pseudoephedrine and dextromethorphan which section 1 has.

Optionally the section 1 is taken at night time and the section 2 is taken in day time.

Optionally the medication does not comprise dextromethorphan.

### Second Aspect—Method of Treatment

Optionally the invention involves a method of treating any one or more of mucosal congestion, coughing, fever, sinus secretions, muscle ache, malaise, cold, flu and headache in a human by administering or taking medication comprising paracetamol, ibuprofen, and any one or both of antihistamine and guaifenesin (or a pharmaceutically acceptable salt, metabolite, or enantiomer of any mentioned aforesaid).

Optionally the antihistamine comprises any one or more of cetirizine, bilastine, loratadine, and fexofenadine (or a pharmaceutically acceptable salt, metabolite, or enantiomer of any mentioned aforesaid).

Optionally the medication comprises any one or more of phenylephrine, pseudoephedrine, and dextromethorphan (or a pharmaceutically acceptable salt, metabolite, or enantiomer of any mentioned aforesaid).

Optionally one or more of the ingredients paracetamol, ibuprofen, guaifenesin, cetirizine, bilastine, loratadine, fexofenadine, phenylephrine, pseudoephedrine and dextromethorphan are combined in a dosage unit.

Optionally one or more of the ingredients paracetamol, ibuprofen, guaifenesin, cetirizine, bilastine, loratadine, fexofenadine, phenylephrine, pseudoephedrine and dextromethorphan are in separate dosage units.

Optionally the medication is for administering or taking in a 24-hour period at least one of the following ingredients in the amounts indicated for that ingredient:
a. paracetamol between 1-4,000 mg;
b. ibuprofen between 1-3,200 mg;
c. guaifenesin between 1-2,400 mg;
d. cetirizine between 1-10 mg;
e. bilastine between 1-100 mg;
f. loratadine between 1-10 mg;
g. fexofenadine between 1-180 mg;
h. phenylephrine between 1-60 mg;
i. pseudoephedrine between 1-240 mg; and
j. dextromethorphan between 0-120 mg.

Optionally the medication is for administering or taking in the 24-hour period at least one of the following ingredients in the amounts indicated for that ingredient:
a. paracetamol between 350-4,000 mg;
b. ibuprofen between 100-600 mg;
c. guaifenesin between 300-2,400 mg;
d. cetirizine between 1-10 mg;
e. bilastine between 5-100 mg;
f. loratadine between 1-10 mg;
g. fexofenadine between 30-180 mg;
h. phenylephrine between 1-40 mg;
i. pseudoephedrine between 60-240 mg; and
j. dextromethorphan between 0-120 mg.

Optionally the medication is for administering or taking in 1-10 dosage units during the 24-hour period.

Optionally the medication is for administering or taking in a 12-hour period at least one of the following ingredients in the amounts indicated for that ingredient:
a. paracetamol between 1-2,000 mg;
b. ibuprofen between 1-600 mg;
c. guaifenesin between 1-1,200 mg;
d. cetirizine between 1-5 mg;
e. bilastine between 1-80 mg;
f. loratadine between 1-5 mg;
g. fexofenadine between 1-90 mg;
h. phenylephrine between 1-30 mg;
i. pseudoephedrine between 1-120 mg; and
j. dextromethorphan between 0-80 mg.

Optionally the medication is for administering or taking in 1-5 dosage units during the 12-hour period.

Optionally the medication is for administering or taking at least one of the following ingredients in the amounts indicated for that ingredient:
a. paracetamol between 300-600 mg;
b. ibuprofen between 100-300 mg;
c. guaifenesin between 80-380 mg;
d. cetirizine between 1-10 mg;
e. bilastine between 1-20 mg;
f. loratadine between 1-10 mg;
g. fexofenadine between 1-200 mg;
h. phenylephrine between 1-20 mg;
i. pseudoephedrine between 5-70 mg; and
j. dextromethorphan between 0-25 mg.

Optionally the medication is for administering or taking at least one of the following ingredients in the amounts indicated for that ingredient:
a. paracetamol between 275-575 mg;
b. ibuprofen between 125-250 mg;
c. guaifenesin between 100-300 mg;
d. cetirizine between 1.5-10 mg;
e. bilastine between 1.5-20 mg;
f. loratadine between 1.5-10 mg;
g. fexofenadine between 20-200 mg;
h. phenylephrine between 1-10 mg;
i. pseudoephedrine between 15-55 mg; and
j. dextromethorphan between 0-20 mg.

Optionally the medication is for administering or taking at least one of the following ingredients in the amounts indicated for that ingredient
a. paracetamol between 425-500 mg;
b. ibuprofen between 125-200 mg;
c. guaifenesin between 100-200 mg;
d. cetirizine between 2-10 mg;
e. bilastine between 4-20 mg;
f. loratadine between 2-10 mg;
g. fexofenadine between 40-180 mg;
h. phenylephrine between 2.5-8 mg;
i. pseudoephedrine between 20-50 mg; and
j. dextromethorphan between 0-10 mg.

Optionally the medication is for administering or taking at least one of the following ingredients in the amounts indicated for that ingredient
a. paracetamol 500 mg;
b. ibuprofen 150 mg;
c. guaifenesin 150 mg;
d. cetirizine 2.5 mg;
e. bilastine 5 mg;
f. loratadine 2.5 mg;
g. fexofenadine 30 mg;
h. phenylephrine 5 mg;
i. pseudoephedrine 30 mg; and
j. dextromethorphan between 0-5 mg.

Optionally the medication further comprises a prodrug.

Optionally the prodrug is a peptide covalently linked to pseudoephedrine.

Optionally the peptide is attached by a hydroxyl group to pseudoephedrine.

Optionally the prodrug is lysine.

Optionally the paracetamol, the ibuprofen and any one or both of the

antihistamine and the guaifenesin (or a pharmaceutically acceptable salt, metabolite, or enantiomer of any mentioned aforesaid) are for administering or taking at the same dosage event, on at least some occasions during the same day.

Optionally the paracetamol and the ibuprofen and any one or both of the antihistamine and the guaifenesin (or a pharmaceutically acceptable salt, metabolite, or enantiomer of any mentioned aforesaid) are for administering or taking at different dosage events, on at least some occasions during the same day.

Optionally the medication is such that the paracetamol and the ibuprofen and any one or both of the antihistamine and the guaifenesin (or a pharmaceutically acceptable salt, metabolite, or enantiomer of any mentioned aforesaid) are for administering or taking in doses comprising of one or more of the following forms: a) tablets, b) capsule, c) aerosol and d) liquid.

Optionally the medication is such that the paracetamol, the ibuprofen and the antihistamine (or a pharmaceutically acceptable salt, metabolite, or enantiomer of any mentioned aforesaid) are for administering or taking in doses comprising one or both of the following forms a) tablets and b) capsules.

Optionally the medication is such that the guaifenesin (or a pharmaceutically acceptable salt, metabolite, or enantiomer thereof) is for administering in doses comprising one or both of the following forms a) liquid and b) aerosol form.

Optionally the medication is contained in a kit with instructions on how to take the medication.

Optionally the kit has two sections such that section 1 contains a combination of any one or more of phenylephrine, pseudoephedrine and dextromethorphan while section 2 does not have the combination of phenylephrine, pseudoephedrine and dextromethorphan which section 1 has.

Optionally the section 1 is taken at night time and the section 2 is taken in day time.

Optionally the medication does not comprise dextromethorphan.

### Third Aspect—Product for Use in Treatment

According to a third aspect, the invention involves a medication comprising paracetamol, ibuprofen, and any one or both of antihistamine and guaifenesin (or a pharmaceutically acceptable salt, metabolite, or enantiomer of any mentioned aforesaid) for use in the treatment of any one or more of mucosal congestion, coughing, fever, sinus secretions, muscle ache, malaise, cold, flu and headache in a human.

Optionally the antihistamine comprises any one or more of cetirizine, bilastine, loratadine, and fexofenadine (or a pharmaceutically acceptable salt, metabolite, or enantiomer of any mentioned aforesaid).

Optionally the medication comprises any one or more of phenylephrine, pseudoephedrine, and dextromethorphan (or a pharmaceutically acceptable salt, metabolite, or enantiomer of any mentioned aforesaid).

Optionally one or more of the ingredients paracetamol, ibuprofen, guaifenesin, cetirizine, bilastine, loratadine, fexofenadine, phenylephrine, pseudoephedrine and dextromethorphan are combined in a dosage unit.

Optionally one or more of the ingredients paracetamol, ibuprofen, guaifenesin, cetirizine, bilastine, loratadine, fexofenadine, phenylephrine, pseudoephedrine and dextromethorphan are in separate dosage units.

Optionally the medication is for administering or taking in a 24-hour period at least one of the following ingredients in the amounts indicated for that ingredient:
a. paracetamol between 1-4,000 mg;
b. ibuprofen between 1-3,200 mg;
c. guaifenesin between 1-2,400 mg;
d. cetirizine between 1-10 mg;
e. bilastine between 1-100 mg;
f. loratadine between 1-10 mg;
g. fexofenadine between 1-180 mg;
h. phenylephrine between 1-60 mg;
i. pseudoephedrine between 1-240 mg; and
j. dextromethorphan between 0-120 mg.

Optionally the medication is for administering or taking in the 24-hour period at least one of the following ingredients in the amounts indicated for that ingredient:
a. paracetamol between 350-4,000 mg;
b. ibuprofen between 100-600 mg;
c. guaifenesin between 300-2,400 mg;
d. cetirizine between 1-10 mg;
e. bilastine between 5-100 mg;
f. loratadine between 1-10 mg;
g. fexofenadine between 30-180 mg;
h. phenylephrine between 1-40 mg;
i. pseudoephedrine between 60-240 mg; and
j. dextromethorphan between 0-120 mg.

Optionally the medication is for administering or taking in 1-10 dosage units during the 24-hour period.

Optionally the medication is for administering or taking in a 12-hour period at least one of the following ingredients in the amounts indicated for that ingredient:
a. paracetamol between 1-2,000 mg;
b. ibuprofen between 1-600 mg;
c. guaifenesin between 1-1,200 mg;
d. cetirizine between 1-5 mg;
e. bilastine between 1-80 mg;
f. loratadine between 1-5 mg;
g. fexofenadine between 1-90 mg;
h. phenylephrine between 1-30 mg;
i. pseudoephedrine between 1-120 mg; and
j. dextromethorphan between 0-80 mg.

Optionally the medication is for administering or taking in 1-5 dosage units during the 12-hour period.

Optionally the medication is for administering or taking at least one of the following ingredients in the amounts indicated for that ingredient:
a. paracetamol between 300-600 mg;
b. ibuprofen between 100-300 mg;
c. guaifenesin between 80-380 mg;
d. cetirizine between 1-10 mg;
e. bilastine between 1-20 mg;
f. loratadine between 1-10 mg;
g. fexofenadine between 1-200 mg;
h. phenylephrine between 1-20 mg;
i. pseudoephedrine between 5-70 mg; and
j. dextromethorphan between 0-25 mg.

Optionally the medication is for administering or taking at least one of the following ingredients in the amounts indicated for that ingredient
a. paracetamol between 275-575 mg;
b. ibuprofen between 125-250 mg;
c. guaifenesin between 100-300 mg;
d. cetirizine between 1.5-10 mg;
e. bilastine between 1.5-20 mg;
f. loratadine between 1.5-10 mg;
g. fexofenadine between 20-200 mg;
h. phenylephrine between 1-10 mg;
i. pseudoephedrine between 15-55 mg; and
j. dextromethorphan between 0-20 mg.

Optionally the medication is for administering or taking at least one of the following ingredients in the amounts indicated for that ingredient
a. paracetamol between 425-500 mg;
b. ibuprofen between 125-200 mg;
c. guaifenesin between 100-200 mg;
d. cetirizine between 2-10 mg;
e. bilastine between 4-20 mg;
f. loratadine between 2-10 mg;
g. fexofenadine between 40-180 mg;
h. phenylephrine between 2.5-8 mg;
i. pseudoephedrine between 20-50 mg; and
j. dextromethorphan between 0-10 mg.

Optionally the medication is for administering or taking at least one of the following ingredients in the amounts indicated for that ingredient
a. paracetamol 500 mg;
b. ibuprofen 150 mg;
c. guaifenesin 150 mg;
d. cetirizine 2.5 mg;
e. bilastine 5 mg;
f. loratadine 2.5 mg;
g. fexofenadine 30 mg;
h. phenylephrine 5 mg;
i. pseudoephedrine 30 mg; and
j. dextromethorphan between 0-5 mg.

Optionally the medication further comprises a prodrug.

Optionally the prodrug is a peptide covalently linked to pseudoephedrine.

Optionally the peptide is attached by a hydroxyl group to pseudoephedrine.

Optionally the prodrug is lysine.

Optionally the paracetamol, the ibuprofen and any one or both of the antihistamine and the guaifenesin (or a pharmaceutically acceptable salt, metabolite, or enantiomer of any mentioned aforesaid) are for administering or taking at the same dosage event, on at least some occasions during the same day.

Optionally the paracetamol and the ibuprofen and any one or both of the antihistamine and the guaifenesin (or a pharmaceutically acceptable salt, metabolite, or enantiomer of any mentioned aforesaid) are for administering or taking at different dosage events, on at least some occasions during the same day.

Optionally the medication is such that the paracetamol and the ibuprofen and any one or both of the antihistamine and the guaifenesin (or a pharmaceutically acceptable salt, metabolite, or enantiomer of any mentioned aforesaid) are for administering or taking in doses comprising of one or more of the following forms: a) tablets, b) capsule, c) aerosol and d) liquid.

Optionally the medication is such that the paracetamol, the ibuprofen and the antihistamine (or a pharmaceutically acceptable salt, metabolite, or enantiomer of any mentioned aforesaid) are for administering or taking in doses comprising one or both of the following forms a) tablets and b) capsules.

Optionally the medication is such that the guaifenesin (or a pharmaceutically acceptable salt, metabolite, or enantiomer thereof is for administering in doses comprising one or both of the following forms a) liquid and b) aerosol form.

Optionally the medication is contained in a kit with instructions on how to take the medication.

Optionally the kit has two sections such that section 1 contains a combination

of any one or more of phenylephrine, pseudoephedrine and dextromethorphan while section 2 does not have the combination of phenylephrine, pseudoephedrine and dextromethorphan which section 1 has.

Optionally the section 1 is taken at night time and the section 2 is taken in day time.

Optionally the medication does not comprise dextromethorphan.

### DETAILED DESCRIPTION

### Relief from GFN when Combined with IBF+APAP

A normal 4 hourly dosage of DM and GFN is 20 mg and 400 mg respectively. The inventor has found that the relief given by the above doses can be achieved by a GFN dose of 280 mg respectively when combined with IBF+APAP. This is because the effectiveness of GFN goes up about 45% when combined with IBF+APAP and because the combination provides the same amount of relief without DM. This was not known before. Therefore, it is suggested, that when 175-200 mg IBF and 275-575 mg APAP is taken with 100 mg GFN then it will lead to a therapeutic relief of 143 mg GFN. Similarly, it is suggested when 175-200 mg IBF and 275-575 mg APAP is taken with 200 mg GFN it will lead to a therapeutic relief of 286 mg GFN. Therefore, a lower amount of GFN when used in combination with IBF+APAP gives a therapeutic effect of a higher amount. In short, the lower efficacy GFN can be offset by a limited extent when used with IBF+APAP.

It is suggested that at DM+GFN dosages of 3 mg-150 mg, and 6 mg-180 mg, the gastric side effects and other side effects are low, the medicine is still therapeutically effective however provides low relief, and DM is in low enough dosage so that it is less likely to be abused and that vendors will be more willing to stock it given that medicines containing high dosages of DM are discouraged. If IBF+APAP is added to the doses, then the therapeutic efficiency further increases to provide high relief without the negative side effects.

The low DM+GFN doses mentioned above are counter intuitive. 10 mg DM is considered hourly equal to dosage of 200 mg GFN. Similarly, 20 mg DM is considered as a 4-hour dosage and is combined with 400 mg GFN which is also a 4 hour dosage. It will be counter intuitive to use different hour dosages of DM and GFN together. For example, a reasonable person will not use 3-6 mg DM (a 1-hour dosage) with 180 mg GFN (near to a 4 hour dosage). Furthermore, the low dosages mentioned become even more counterintuitive when combined with the varying hourly dosages of IBF, APAP, and antihistamines mentioned in this specification.

While the exact pathway of GFN action is not known, the suggested reasoning behind the improvement is that GFN is lost during metabolism in the gastrointestinal tract and in the liver which limits bioavailability. IBF+APAP leads to an increase in competition which results in more GFN making it to the blood stream. It is found that the method of administration also leads to positive results. Low dosages result in a flat peak and trough concentration in the blood plasma. A high difference between peak and trough concentrations in the plasma is considered to have more negative effects. Low doses with high frequency of administration maximises the advantages provided by the flat peak and trough concentration. Because IBF+APAP enhances the effect of GFN and overall relief, lower amounts of GFN provide better results, reduces/omits the need to use DM in medicines and decreases negative side effects (e.g gastric discomfort, dizziness, etc) due to taking high dosages of DM and GFN.

### Relief from Antihistamines when Combined with APAP+IBF

The inventor has found that effectiveness for antihistamines (e.g., cetirizine, bilastine, loratadine, fexofenadine etc) is increased by about 25% to about 50% when taken with APAP and IBF. It has also been found that antihistamines when taken with APAP and IBF led to higher relief than medicines consisting of DM+GFN. This has not been known before.

While the full pathway for APAP is not known, it is suggested that APAP allows more of IBF and antihistamines to get through to the blood stream and limits the disadvantages (as discussed previously) from the metabolism in the liver and gastrointestinal tract. Secondly, when lower doses of APAP, IBF and antihistamines are taken with high frequency of administration then this maximises the advantages (as discussed previously) provided by the flat peak and trough concentration in the blood plasma. Therefore, the efficiency of the active ingredients is increased and furthermore is cost effective as less amount results in more relief.

By way of example, it has been found that about 2.5 mg cetirizine, 5 mg bilastine, 2.5 mg loratadine, or 30 mg fexofenadine administered 4 times daily have better effect than 10 mg cetirizine, 20 mg bilastine, 10 mg loratadine or 120 mg fexofenadine administered once. This type of administration resembles continuous infusion as there is always a constant concentration of antihistamine over time. A constant low concentration of antihistamine is more advantageous that a high antihistamine dose which results in a sudden rise and drop in concentration. A sudden rise in drug concentration leads to increase in negative side effects of the drug while a big drop in concentration results in the body to make increased adjustments to maintain a constant homeostasis which also overall negatively effects health.

It is suggested that antihistamines provide relief to more symptoms than discovered. Recently, it has came to light that antihistamines provide relief for long Covid symptoms (Reference: Melissa D. Pinto, Natalie Lambert, Charles A. Downs, Heather Abrahim, Thomas D. Hughes, Amir M. Rahmani, Candace W. Burton, Rana Chakraborty, Antihistamines for Postacute Sequelae of SARS-COV-2 Infection, The Journal for Nurse Practitioners, Volume 18, Issue 3, 2022, Pages 335-338).

Histamine plays a big role in homeostasis as its receptors are located throughout the body. According to a research paper by Tatarkiewicz, J et al:

“Histamine is involved in numerous physiological and pathophysiological processes [6]—immunological response, immunomodulation, inflammation, allergic response, gastric acid secretion, cell proliferation, wound healing, cognitive function, memory, sleep cycle, endocrine homeostasis—and has an influence on release of other neurotransmitters [7] and modulation of tumor growth” (Reference: Tatarkiewicz, J., Rzodkiewicz, P., Żochowska, M., Staniszewska, A., & Bujalska-Zadrożny, M. (2019). New antihistamines—perspectives in the treatment of some allergic and inflammatory disorders. Archives of medical science: AMS, 15(2), 537-553).

It is suggested that antihistamines have more influence than currently known due to the placement of H1 receptors and histamine being responsible for a wide range of symptoms. The inventor has found that antihistamines (e.g., cetirizine, bilastine, loratadine, fexofenadine etc) are enhanced by APAP+IBF, such that they can be used to decrease/omit the usage of DM and GFN in medication.

The inventor developed combinations comprising 500 mg APAP+150 mg IBF +any one or more of 175 mg GFN, 2.5 mg cetirizine, 5 mg bilastine, 2.5 mg loratadine and 30 mg fexofenadine. The stated amounts for APAP, IBF and GFN are considered about or near to 4 hour doses while the amounts for antihistamine are considered about or near to 6 hours doses. It will be counter intuitive to use different hour dosages of ingredients together as a reasonable person skilled in the art will use equal hour dosages.

### Relief from Antihistamines+PE+(APAP and/or IBF)

The inventor has found that effectiveness for antihistamines (e.g., cetirizine, bilastine, loratadine, fexofenadine etc) is increased by about 10% to about 30% when taken with PE and APAP and/or IBF. This has not been known before. It has also been found that antihistamines when taken with PE+APAP and/or IBF led to higher relief than medicines consisting of DM+GFN.

As stated previously, this is surprising because DM is a cough suppressant and GFN is an expectorant while the other ingredients (e.g., cetirizine, bilastine, loratadine, fexofenadine, APAP, PE and IBF) are either a decongestant, antihistamine(s), or painkillers and not expected to fully replace a cough suppressant and an expectorant.

As stated previously, the benefit of the PE+antihistamine+APAP and/or IBF combination is that less amount of PE and antihistamine has to be used to give the therapeutic effect of a higher amount, the combination omits or decreases the use of DM and GFN, the combination will decrease drug abuse, vendors are more likely to stock it as OTC medication due to decreased negative effects and government restrictions and therefore more available to those in dire need of the medication.

The inventor developed combinations comprising 5 mg PE+any one of 2.5 mg cetirizine, 5 mg bilastine, 2.5 mg loratadine and 30 mg fexofenadine+500 mg APAP and/or 150 mg IBF. The stated amounts for APAP and IBF are considered about or near to 4 hour doses, amount stated for PE is about a 2 hour dose while the amounts stated for antihistamine are considered about or near to 6 hours doses. It will be counter intuitive to use different hour dosages of ingredients together as a reasonable person skilled in the art will use equal hour dosages.

### Preferred Forms

Other non-active ingredients such as binders and excipients, known to the person skilled in the art, are intended to be included into the preferred composition. The active ingredients can be formulated into a liquid, pill, tablet or capsule using common pharmaceutical carriers and excipients. The active ingredients can be administered either together or separately at the same or different times during the day. The average preferable particle size for each active ingredient is to be between 1-16μm or 25-85 μm. The most preferrable average particle size for each active ingredient is either 6±5 μm or 25±5 μm or 45±5 μm or 60±5 μm. It is suggested that the dissolution is most effective where the average particle size is either 6 μm or 25 μm or 45 μm or 55 μm.

In another embodiment the pharmaceutical composition can be taken as a syrup for patients who are children or have difficulty in swallowing pills. The standard production method of syrup is very well known in the art. The syrup may be in any container (e.g. vial, bottle etc). It is regarded as appropriate to serve lower doses for children. The viscosity of the syrup is preferred to be between 900 cpm to 2500 cpm. The most preferable viscosity is 900±50 cpm and 2000±100 cpm. The preferred pH maintained by the buffering agent in the syrup is below 5. High level of sorbitol and maltose in syrups are known to contribute to faster degradation of the active ingredients, therefore for a palatable flavour and decreased degradation, the syrup preferably consists of artificial flavour, sorbitol up to 12% w/v, glycerine up to 48% w/v where the ratio of glycerine to sorbitol is 3:1 to 11:1.

In another embodiment the pharmaceutical composition can be taken as a nasal spray (preferably aerosol form). The standard production method of a nasal (aerosol) spray is very well known in the art.

Preferably, the pharmaceutical composition is provided in a kit with instructions on how to take it. Preferably, the kit has two sections where section 1 is to be taken in the day and section 2 is to be taken at night. The main difference between the sections is that section 2 contains one or more of phenylephrine, pseudoephedrine and dextromethorphan while section 1 does not have the combination of phenylephrine, pseudoephedrine and dextromethorphan which section 2 has. In an alternative embodiment, the kit may entirely consist of either section 1 or 2.

TABLE 4
Examples of Compositions

| Example 1 | Example 2 | Example 3 |
|---|---|---|
| (Tablet) | (Syrup) | (Capsule) |
| Active ingredients | Active ingredients | Active ingredients |
| APAP | APAP | APAP |
| IBF | IBF | IBF |
| GFN | GFN | GFN |
| antihistamines | antihistamines | antihistamines |
| (e.g., cetirizine, | (e.g., cetirizine, | (e.g., cetirizine, |
| bilastine, loratadine, | bilastine, loratadine, | bilastine, loratadine, |
| fexofenadine etc) | fexofenadine etc) | fexofenadine etc) |
| PE, PSE or PSE + | PE, PSE or PSE + | PE, PSE or PSE + |
| lysine | lysine | lysine |
| Dextromethorphan | Dextromethorphan | Dextromethorphan |
| Inactive ingredients | Inactive ingredients | Inactive ingredients |
| Silicon Dioxide | Edetate Disodium | Gelatin |
| Croscarmellose Sodium | Fd&C Red No. 4 0 | Propylene Glycol |
| Crospovidone | Glycerin | Sorbitol Solution |
| Fd&C Red No 40 | Menthol, | Water |
| | Unspecified Form | |
| Aluminum Oxide | Polyethylene Glycol, | Titanium Dioxide |
| FD & C Yellow No 6 | Propyl Gallate | FD & C Yellow No. 6 |
| Magnesium Stearate | Propylene Glycol | Glycerin |
| Maltodextrin | Water | Polyethylene Glycol |
| Cellulose, | Sodium Benzoate | Povidone |
| Microcrystalline | | |
| Polyethylene Glycol, | Sodium Citrate, | |
| Polyvinyl Alcohol, | Sorbitol | |
| Povidone | Sucralose | |
| Starch, Corn | Triacetin | |
| Stearic Acid | Xanthan Gum | |
| Talc | Anhydrous Citric Acid | |
| Titanium Dioxide | | |

### Clinical Study

The subject had recurring coughs, sneezing, headache, runny nose, pain, cold, fever, sore throat, sinus pressure etc. They took medication including DM+GFN combination as well as antihistamines to cure it, namely:
a. Mucinex Expectorant Tablets (600 mg GFN/dose): 1 dose per 12 hours;
b. Robitussin Cough and Chest Congestion syrup (200 mg GFN+30 mg DM/dose): 1 dose per 6 hours for total 12 hours;
c. Maxiclear Sinus Relief (10 mg Phenylephrine hydrochloride/dose): 1 dose per 4 hours for total 12 hours;
d. Cetirizine Hayfever & Allergy Relief (10 mg Cetirizine hydrochloride/dose): 1 dose per 24 hours;
e. Loraclear (10 mg Loratidine/dose): 1 dose per 24 hours;
f. Telfast (120 mg Fexofenadine Hydrochloride/dose): 1 dose per 24 hours; and
g. Labixten (20 mg Bilastine/dose): 1 dose per 24 hours.

The subject was given new pharmaceutical combinations and did not take any other cough, cold, allergy, pain or flu medications during the duration of the study. They rated improvement from the new combination and compared relief with their previous medication (mentioned above) by stating the overall effect and/or giving a percentage score between 0-100%.

The invention is not to be limited by any embodiments described here. Various modification will be apparent to those skilled in the art and are intended to fall within the scope of the claims. For example, variation in dosages or modification in the method of administering the invention are also sought to be covered by the invention. Example of some dosages which can be prepared are shown below

| | | | Amount of doses/ |
|---|---|---|---|
| Block No. | Name | Amount per dose | day or hours |
| 1 | DM + | 3 mg-150 mg-450 mg, | up to 10 doses/day |
| | IBF + | 3 mg-180 mg-450 mg, | or until maximum |
| | APAP | 3 mg-200 mg-450 mg, | daily dosage |
| | | 6 mg-150 mg-450 mg, | allowed is achieved |
| | | 6 mg-180 mg-450 mg, | for any drug. |
| | | 6 mg-200 mg-450 mg, | Administration |
| | | 10 mg-180 mg-450 mg, | should stop on |
| | | 12 mg-150 mg-450 mg, | whichever option is |
| | | 12 mg-180 mg-450 mg, | fulfilled first. |
| | | 12 mg-200 mg-450 mg, | |
| | | 15 mg-150 mg-450 mg, | |
| | | 15 mg-180 mg-450 mg | |
| | | &15 mg-200 mg-450 mg. | |
| 2 | GFN + | 90 mg-150 mg-450 mg, | up to 10 doses/day |
| | IBF + | 120 mg-150 mg-450 mg, | or until maximum |
| | APAP | 150 mg-150 mg-450 mg, | daily dosage |
| | | 150 mg-180 mg-450 mg, | allowed is achieved |
| | | 150 mg-200 mg-450 mg, | for any drug. |
| | | 170 mg-150 mg-450 mg, | Administration |
| | | 170 mg-180 mg-450 mg, | should stop on |
| | | 170 mg-200 mg-450 mg, | whichever option is |
| | | 180 mg-150 mg-450 mg, | fulfilled first. |
| | | 180 mg-180 mg-450 mg, | |
| | | 180 mg-200 mg-450 mg, | |
| | | 190 mg-150 mg-450 mg, | |
| | | 190 mg-180 mg-450 mg, | |
| | | 190 mg-200 mg-450 mg, | |
| | | 200 mg-150 mg-450 mg, | |
| | | 200 mg-180 mg-450 mg, | |
| | | & 200 mg-200 mg-450 mg. | |
| 3 | DM + | 3 mg-150 mg-150 mg-450 mg, | up to 10 doses/day |
| | GFN + | 6 mg-150 mg-150 mg-450 mg, | or until maximum |
| | IBF + | 3 mg-160 mg-150 mg-450 mg, | daily dosage |
| | APAP | 3 mg-170 mg-150 mg-450 mg, | allowed is achieved |
| | | 3 mg-180 mg-150 mg-450 mg, | for any drug. |
| | | 6 mg-180 mg-150 mg-450 mg, | Administration |
| | | 3 mg-190 mg-150 mg-450 mg, | should stop on |
| | | 6 mg-190 mg-150 mg-450 mg, | whichever option is |
| | | 3 mg-200 mg-150 mg-450 mg, | fulfilled first. |
| | | 6 mg-200 mg-150 mg-450 mg, | |
| | | 6 mg-220 mg-150 mg-450 mg, | |
| | | 6 mg-230 mg-150 mg-450 mg, | |
| | | 6 mg-240 mg-150 mg-450 mg, | |
| | | 6 mg-250 mg-150 mg-450 mg, | |
| | | 3 mg-150 mg-180 mg-450 mg, | |
| | | 6 mg-150 mg-180 mg-450 mg, | |
| | | 3 mg-160 mg-180 mg-450 mg, | |
| | | 3 mg-170 mg-180 mg-450 mg, | |
| | | 3 mg-180 mg-180 mg-450 mg, | |
| | | 6 mg-180 mg-180 mg-450 mg, | |
| | | 3 mg-190 mg-180 mg-450 mg, | |
| | | 6 mg-190 mg-180 mg-450 mg, | |
| | | 3 mg-200 mg-180 mg-450 mg, | |
| | | 6 mg-200 mg-180 mg-450 mg, | |
| | | 6 mg-220 mg-180 mg-450 mg, | |
| | | 6 mg-230 mg-180 mg-450 mg, | |
| | | 6 mg-240 mg-180 mg-450 mg, | |
| | | 6 mg-250 mg-180 mg-450 mg, | |
| | | 3 mg-150 mg-200 mg-450 mg, | |
| | | 6 mg-150 mg-200 mg-450 mg, | |
| | | 3 mg-160 mg-200 mg-450 mg, | |
| | | 3 mg-170 mg-200 mg-450 mg, | |
| | | 3 mg-180 mg-200 mg-450 mg, | |
| | | 6 mg-180 mg-200 mg-450 mg, | |
| | | 3 mg-190 mg-200 mg-450 mg, | |
| | | 6 mg-190 mg-200 mg-450 mg, | |
| | | 3 mg-200 mg-200 mg-450 mg, | |
| | | 6 mg-200 mg-200 mg-450 mg, | |
| | | 6 mg-220 mg-200 mg-450 mg, | |
| | | 6 mg-230 mg-200 mg-450 mg, | |
| | | 6 mg-240 mg-200 mg-450 mg | |
| | | & 6 mg-250 mg-200 mg-450 mg. | |
| 4 | APAP + | 450 mg-150 mg-2.5 mg, | up to 4 doses/12 hours |
| | IBF + | 500 mg-150 mg-2.5 mg, | or until maximum |
| | cetirizine | 450 mg-175 mg-5 mg, | daily dosage |
| | | 500 mg-175 mg-5 mg, | allowed is achieved |
| | | 450 mg-200 mg-10 mg, | for any drug. |
| | | 500 mg-200 mg-10 mg | Administration |
| | | | should stop on |
| | | | whichever option is |
| | | | fulfilled first. |
| 5 | APAP + | 450 mg-150 mg-5 mg, | up to 4 doses/12 hours |
| | IBF + | 500 mg-150 mg-5 mg, | or until maximum |
| | bilastine | 450 mg-175 mg-10 mg, | daily dosage |
| | | 500 mg-175 mg-10 mg, | allowed is achieved |
| | | 450 mg-200 mg-20 mg, | for any drug. |
| | | 500 mg-200 mg-20 mg | Administration |
| | | | should stop on |
| | | | whichever option is |
| | | | fulfilled first. |
| 6 | APAP + | 450 mg-150 mg-2.5 mg, | up to 4 doses/12 hours |
| | IBF + | 500 mg-150 mg-2.5 mg, | or until maximum |
| | loratadine | 450 mg-175 mg-5 mg, | daily dosage |
| | | 500 mg-175 mg-5 mg, | allowed is achieved |
| | | 450 mg-200 mg-10 mg, | for any drug. |
| | | 500 mg-200 mg-10 mg | Administration |
| | | | should stop on |
| | | | whichever option is |
| | | | fulfilled first. |
| 7 | APAP + | 450 mg-150 mg-1.25 mg-1.25 mg, | up to 4 doses/12 hours |
| | IBF + | 500 mg-150 mg-1.25 mg-1.25 mg, | or until maximum |
| | cetirizine + | 450 mg-175 mg-1.5 mg-1.5 mg, | daily dosage |
| | loratadine | 500 mg-175 mg-1.5 mg-1.5 mg, | allowed is achieved |
| | | 450 mg-200 mg-2.5 mg-2.5 mg, | for any drug. |
| | | 500 mg-200 mg-2.5 mg-2.5 mg, | Administration |
| | | 450 mg-200 mg-5 mg-5 mg, | should stop on |
| | | 500 mg-200 mg-5 mg-5 mg | whichever option is |
| | | | fulfilled first. |
| 8 | APAP + | 450 mg-150 mg-30 mg, | up to 4 doses/12 hours |
| | IBF + | 500 mg-150 mg-30 mg, | or until maximum |
| | fexofenadine | 450 mg-175 mg-60 mg, | daily dosage |
| | | 500 mg-175 mg-60 mg, | allowed is achieved |
| | | 450 mg-200 mg-180 mg, | for any drug. |
| | | 500 mg-200 mg-180 mg | Administration |
| | | | should stop on |
| | | | whichever option is |
| | | | fulfilled first. |
| 9 | APAP + | 450 mg-150 mg-150 mg-2.5 mg, | up to 4 doses/12 hours |
| | GFN + | 500 mg-150 mg-150 mg-2.5 mg, | or until maximum |
| | IBF + | 450 mg-175 mg-175 mg-5 mg, | daily dosage |
| | cetirizine | 500 mg-175 mg-175 mg-5 mg, | allowed is achieved |
| | | 450 mg-200 mg-200 mg-10 mg, | for any drug. |
| | | 500 mg-200 mg-200 mg-10 mg, | Administration |
| | | | should stop on |
| | | | whichever option is |
| | | | fulfilled first. |
| 10 | APAP + | 450 mg-150 mg-150 mg-5 mg, | up to 4 doses/12 hours |
| | GFN + | 500 mg-150 mg-150 mg-5 mg, | or until maximum |
| | IBF + | 450 mg-175 mg-175 mg-10 mg, | daily dosage |
| | bilastine | 500 mg-175 mg-175 mg-10 mg, | allowed is achieved |
| | | 450 mg-200 mg-200 mg-20 mg, | for any drug. |
| | | 500 mg-200 mg-200 mg-20 mg, | Administration |
| | | | should stop on |
| | | | whichever option is |
| | | | fulfilled first. |
| 11 | APAP + | 450 mg-150 mg-150 mg-2.5 mg, | up to 4 doses/12 hours |
| | GFN + | 500 mg-150 mg-150 mg-2.5 mg, | or until maximum |
| | IBF + | 450 mg-175 mg-175 mg-5 mg, | daily dosage |
| | loratadine | 500 mg-175 mg-175 mg-5 mg, | allowed is achieved |
| | | 450 mg-200 mg-200 mg-10 mg, | for any drug. |
| | | 500 mg-200 mg-200 mg-10 mg | Administration |
| | | | should stop on |
| | | | whichever option is |
| | | | fulfilled first. |
| 12 | APAP + | 450 mg-150 mg-150 mg-1.25 mg-1.25 mg, | up to 4 doses/12 hours |
| | GFN + | 500 mg-150 mg-150 mg-1.25 mg-1.25 mg, | or until maximum |
| | IBF + | 450 mg-175 mg-175 mg-1.5 mg-1.5 mg, | daily dosage |
| | cetirizine + | 500 mg-175 mg-175 mg-1.5 mg-1.5 mg, | allowed is achieved |
| | loratadine | 450 mg-200 mg-200 mg-2.5 mg-2.5 mg, | for any drug. |
| | | 500 mg-200 mg-200 mg-2.5 mg-2.5 mg, | Administration |
| | | 450 mg-200 mg-200 mg-5 mg-5 mg, | should stop on |
| | | 500 mg-200 mg-200 mg-5 mg-5 mg | whichever option is |
| | | | fulfilled first. |
| 13 | APAP + | 450 mg-150 mg-150 mg-30 mg, | up to 4 doses/12 hours |
| | GFN + | 500 mg-150 mg-150 mg-30 mg, | or until maximum |
| | IBF + | 450 mg-175 mg-175 mg-60 mg, | daily dosage |
| | fexofenadine | 500 mg-175 mg-175 mg-60 mg, | allowed is achieved |
| | | 450 mg-200 mg-200 mg-180 mg, | for any drug. |
| | | 500 mg-200 mg-200 mg-180 mg | Administration |
| | | | should stop on |
| | | | whichever option is |
| | | | fulfilled first. |
| 14 | PE + | 1-20 mg PE + any of the | up to 4 doses/12 hours |
| | anthistamine + | above doses mentioned for | or until maximum |
| | APAP | any of the above | daily dosage |
| | and/or IBF | antihistamines + any of | allowed is achieved |
| | | the above doses mentioned | for any drug. |
| | | for APAP and/or IBF. | Administration |
| | | | should stop on |
| | | | whichever option is |
| | | | fulfilled first. |

The subject was given 4 doses of each new pharmaceutical combination, shown in the examples below, where each dose was administered once after every 3 hours for a total period of 12 hours. On each recurrence of cough and other symptoms a new pharmaceutical combination was given.

The subject would then provide feedback on the improvement of their symptoms and rate the performance of the pharmaceutical composition in comparison to their previous medication experience.

### Example 4

Pharmaceutical combination 1: GFN and IBF and APAP: 130 mg-150 mg-500 mg respectively.

Results: With the recommended dose, the subject reported about 25% better recovery with decreased gastric side effects in comparison to Mucinex Expectorant Tablets. Overall, there was an increased reduction in coughing, headache and congestion.

### Example 5

Pharmaceutical combination 2: APAP and IBF and cetirizine: 500 mg-150 mg-2.5 mg respectively.

Results: With the new combination, the subject reported better recovery in comparison to Robitussin Cough and Chest Congestion syrup and about 50% better recovery in comparison to Cetirizine Hayfever & Allergy Relief. Overall, there was an increased reduction in coughing, gastric problems, fever, cold, headache and congestion.

### Example 6

Pharmaceutical combination 3: APAP and IBF and GFN and cetirizine: 500 mg-150 mg-175 mg-2.5 mg respectively.

Results: With the new combination, the subject reported better recovery in comparison to Robitussin Cough and Chest Congestion syrup. Overall, there was an increased reduction in dizziness, gastric problems, coughing, fever, and congestion.

### Example 7

Pharmaceutical combination 4: APAP and IBF and loratadine: 500 mg-150 mg-2.5 mg respectively.

Results: With the new combination, the subject reported better recovery in comparison to Robitussin Cough and Chest Congestion syrup and about 45% better recovery in comparison to Loraclear. Overall, there was an increased reduction in dizziness, gastric problems, cold, headache and congestion.

### Example 8

Pharmaceutical combination 5: APAP and IBF and GFN and loratadine: 500 mg-150 mg-175 mg-2.5 mg respectively.

Results: With the new combination, the subject reported better recovery in comparison to Robitussin Cough and Chest Congestion syrup. Overall, there was an increased reduction in dizziness, gastric problems, coughing, headache and congestion.

### Example 9

Pharmaceutical combination 6: APAP and IBF and fexofenadine: 500 mg-150 mg-30 mg respectively.

Results: With the new combination, the subject reported better recovery in comparison to Robitussin Cough and Chest Congestion syrup and about 35% better recovery in comparison to Telfast. Overall, there was an increased reduction in dizziness, gastric problems, coughing, fever, cold, headache and congestion in comparison to their current medicine regime.

### Example 10

Pharmaceutical combination 7: APAP and IBF and GFN and fexofenadine: 500 mg-150 mg-175 mg-30 mg respectively.

Results: With the new combination, the subject reported better recovery in comparison to Robitussin Cough and Chest Congestion syrup. Overall, there was an increased reduction in dizziness, fever, cold, headache and congestion in comparison to their current medicine regime.

### Example 11

Pharmaceutical combination 8: APAP and IBF and Bilastine: 500 mg-150 mg-5 mg respectively.

Results: With the new combination, the subject reported better recovery in comparison to Robitussin Cough and Chest Congestion syrup and about 40% better recovery in comparison to Labixten. Overall, there was an increased reduction in dizziness, gastric problems, gastric problems, cold, headache and congestion.

### Example 12

Pharmaceutical combination 9: APAP and IBF and GFN and Bilastine: 500 mg-150 mg-175 mg-5 mg respectively.

Results: With the new combination, the subject reported better recovery in comparison to Robitussin Cough and Chest Congestion syrup. Overall, there was an increased reduction in dizziness, gastric problems, coughing, headache and congestion.

### Example 13

Any one of 2.5 mg cetirizine, 5 mg bilastine, 2.5 mg loratadine and 30 mg fexofenadine taken in 24 hours.

The subjected reported that any one of 2.5 mg cetirizine, 5 mg bilastine, 2.5 mg loratadine, and 30 mg fexofenadine administered 4 times (each dose after 3 hours) daily performed therapeutically better in comparison to relief gained from taking a normal full 24-hour dose of the same ingredient at once. The subject took the normal full 24-hour dose by using brands namely, Cetirizine Hayfever & Allergy Relief, Loraclear, Telfast and Labixten.

### Example 14

Pharmaceutical combination 10: antihistamine (any one of 2.5 mg cetirizine, 5 mg bilastine, 2.5 mg loratadine and 30 mg fexofenadine)+5 mg PE.

The subjected reported above combinations to perform therapeutically better in comparison to relief gained from individually taking Maxiclear Sinus Relief and antihistamine medications (e.g., Mucinex Expectorant Tablets, Cetirizine Hayfever & Allergy Relief, Loraclear, Telfast and Labixten).

### Example 15

Pharmaceutical combination 11: (500 mg APAP and/or 150 mg IBF)+antihistamine (any one of 2.5 mg cetirizine, 5 mg bilastine, 2.5 mg loratadine and 30 mg fexofenadine)+5 mg PE.

The subjected reported above combinations to perform therapeutically better in comparison to relief gained from a) taking Robitussin Cough and Chest Congestion syrup or b) taking a 24 hour dose antihistamine medication (any one of Mucinex Expectorant Tablets, Cetirizine Hayfever & Allergy Relief, Loraclear, Telfast and Labixten) together with a 24 hour dose of Maxiclear Sinus Relief.

The subject reported about 10%-30% better recovery with the pharmaceutical combination 11 in comparison to antihistamine medication (any one of Mucinex Expectorant Tablets, Cetirizine Hayfever & Allergy Relief, Loraclear, Telfast and Labixten) taken alone.

The subject reported overall significant increased reduction in mucosal congestion, coughing, fever, sinus secretions, muscle ache, malaise, cold, flu, gastric problems and headache.

### Preferred Forms and Embodiments

Some preferred forms of the invention will now be described by way of example. In a preferred embodiment there is a pharmaceutical combination where, IBF, APAP and any one or more of antihistamine(s) and guaifenesin are combined together.

In another preferred embodiment there is a pharmaceutical combination where, antihistamine(s), PE and any one or more of APAP and IBF are combined together.

In another embodiment, IBF, APAP and any one or more of antihistamine(s) and guaifenesin are combined together without the need for DM.

In another embodiment there is a pharmaceutical combination where, antihistamine(s), PE and any one or more of APAP and IBF are combined together without the need for DM and/or GFN.

In another embodiment, APAP, IBF, and antihistamine(s) are combined without the need for DM and/or GFN.

In another embodiment, APAP, GFN, IBF and any one or more of PSE+peptide (e.g., lysine) and PE are combined together.

In another embodiment, APAP, GFN, IBF, any one or more of cetirizine, bilastine, loratadine and fexofenadine, and any one or more of PSE+peptide (e.g., lysine), and PE are combined together.

In another embodiment, 275-575 mg APAP, 125-250 mg IBF, and any one or more of 1-10 mg cetirizine, 1-20 mg bilastine, 1-10 mg loratadine and 30-200 mg fexofenadine are combined together.

In another embodiment, 275-575 mg APAP, 125-250 mg IBF, any one or more of 1-10 mg cetirizine, 1-20 mg bilastine, 1-10 mg loratadine and 30-200 mg fexofenadine, and any one or more of 30-240 mg PSE +peptide (e.g., lysine) and 1-60 mg PE are combined together.

In another embodiment there is a pharmaceutical composition where 275-575 mg APAP, 80-380 mg GFN and 125-250 mg IBF are combined together.

Most preferably the dosage content of APAP, GFN and IBF is 400 mg-500 mg, 150 mg-180 mg and 150 mg-200 mg respectively. In another preferred embodiment the dosage content of APAP, GFN and IBF is 450 mg, 180 mg and 175-200 mg.

In another embodiment 0-15 mg DM, 80-280 mg or 350-380 mg GFN, 275-575 mg APAP, 125-250 mg IBF, any one or more of 1-10 mg cetirizine, 1-20 mg bilastine, 1-10 mg loratadine and 30-200 mg fexofenadine, and any one or more of 30-240 mg PSE+peptide (e.g., lysine) and 1-60 mg PE are combined together.

### Dosages

The dosages can be taken till the maximum daily dose limit is achieved for any one of DM, GFN, APAP, IBF, PE, PSE, PSE+lysine, and antihistamine(s) (e.g., cetirizine, bilastine, loratadine, fexofenadine etc). Alternatively, the medication can be taken at double the dosage to reduce the frequency of administration. That said, it is preferred that the medication is taken in low dosages (e.g half dosages) and increased, evenly spaced, frequency of administration so that there is an optimum level of drug efficiency and relief. The active ingredients can each be administered in the same or different forms (e.g., capsule, tablet or liquid) either together or separately at the same or different times during the day. The dose can be further divided into multiple units. Preferably between 2-6 units. Therefore, instead of taking one big capsule, tablet, or liquid dose the user can take the dose in smaller units which allows easier swallowing. Each unit can comprise of one of more ingredient(s) and can be liquid or solid.

Preferably 1-10 doses can be taken in 24 hours. The maximum 24-hour intake amount for APAP is till 4,000 mg, IBF is till 3,200 mg, GFN is till 2,400 mg, cetirizine is till 10 mg, bilastine is till 100 mg, loratadine is till 10 mg, fexofenadine is till 180 mg, PE is till 60 mg, PSE is till 240 mg and DM is till 120 mg.

Preferably 1-4 doses can be taken in 12 hours. The maximum 12-hour intake amount for APAP is till 2,000 mg, IBF is till 600 mg, GFN is till 1,200 mg, cetirizine is till 5 mg, bilastine is till 80 mg, loratadine is till 5 mg, fexofenadine is till 90 mg, PE is till 30 mg, PSE is till 120 mg and DM is till 80 mg.

Preferably a dose of paracetamol is between 300-600 mg, ibuprofen is between 100-300 mg, guaifenesin is between 80-380 mg, cetirizine is between 1-10 mg, bilastine is between 1-20 mg, loratadine is between 1-10 mg, fexofenadine is between 1-200 mg, phenylephrine is between 1-40 mg, pseudoephedrine is between 5-70 mg and dextromethorphan is between 1-25 mg.

In another embodiment it is preferred that a dose of paracetamol is between 275-575 mg, ibuprofen is between 125-250 mg, guaifenesin is between 100-300 mg, cetirizine is between 1.5-10 mg, bilastine is between 1.5-20 mg, loratadine is between 1.5-10 mg, fexofenadine is between 20-200 mg, phenylephrine is between 1-10 mg, pseudoephedrine is between 15-55 mg and dextromethorphan is between 0-20 mg.

In another embodiment it is preferred that a dose of paracetamol is between 425-500 mg, ibuprofen is between 125-200 mg, guaifenesin is between 100-200 mg, cetirizine is between 2-10 mg, bilastine is between 4-25 mg, loratadine is between 2-10 mg, fexofenadine is between 40-180 mg, phenylephrine is between 2.8-8 mg, pseudoephedrine is between 20-50 mg and dextromethorphan is between 0-10 mg.

In a further embodiment it is preferred that a dose of paracetamol is 500 mg, ibuprofen is 150 mg, guaifenesin is 150 mg, cetirizine is 2.5 mg, bilastine is 5 mg, loratadine is 2.5 mg, fexofenadine is 30 mg, phenylephrine is 5 mg, pseudoephedrine is 30 mg and dextromethorphan is between 0-5 mg.

### Alternative Chemical Forms

DM, GFN, PE, PSE, PSE+lysine, APAP, IBF, cetirizine, bilastine, loratadine and fexofenadine have been referred to directly in this specification. Other acceptable pharmaceuticals forms (e.g salts, enantiomers etc) may also be used and are intended to be captured by the references to the active ingredients with the weight amount adjusted accordingly. Non-limiting examples of enantiomers (in this case of cetirizine) include levocetirizine and dextrocetirizine, and are intended to be captured by the specification. Other acceptable pharmaceutical precursors which lead to the active ingredient before or after metabolization may also be used and are intended to be captured by the references to the active ingredients with the weight amount adjusted accordingly. A non-limiting example of an active ingredient (loratadine) in its metabolized form is desloratadine and is also intended to be captured by the specification.

Other antihistamines, in particular non-sedating antihistamines such as acrivastine, antazoline, astemizole, azelastine, ebastine, efletirizine, epinastine, levocabastine, mizolastine, oxatomide, setastine, temelastine, and terfenadine, are also intended to be captured by the references to antihistamine in this specification.

While some preferred embodiments of the invention have been described by way of example it should be appreciated that modifications and improvements can occur without departing from the scope of the appended claims.

In terms of disclosure, this document envisages and hereby posits any feature mentioned herein in combination with itself or any other feature or features mentioned herein, even if the combination is not claimed.

1. A method of treating or relieving any one or more of mucosal congestion, coughing, sinus secretions, dizziness, and nausea in a human by administering medication comprising paracetamol, ibuprofen, and a non-sedative antihistamine (or a pharmaceutically acceptable salt, or enantiomer of any mentioned aforesaid).

2. The method according to claim 1, wherein the non-sedative antihistamine comprises any one or more of cetirizine, bilastine, loratadine, and fexofenadine (or a pharmaceutically acceptable salt, or enantiomer of any mentioned aforesaid).

3. The method according to claim 2, wherein the medication comprises any one or more of guaifenesin, dextromethorphan, phenylephrine and pseudoephedrine (or a pharmaceutically acceptable salt, or enantiomer of any mentioned aforesaid).

4. The method according to claim 3, wherein one or more of the ingredients paracetamol, ibuprofen, cetirizine, bilastine, loratadine, fexofenadine, guaifenesin, dextromethorphan, phenylephrine and pseudoephedrine are combined in a dosage unit.

5. The method according to claim 3, wherein one or more of the ingredients paracetamol, ibuprofen, cetirizine, bilastine, loratadine, fexofenadine, guaifenesin, dextromethorphan, phenylephrine, and pseudoephedrine are in separate dosage units.

6. The method according to claim 3, wherein the medication is for administering in single or multiple dosage units during a 24-hour period, wherein at least one of the following ingredients is in the amounts indicated for that ingredient:
a) paracetamol between 1-4,000 mg or 350-4,000 mg;
b) ibuprofen between 1-3,200 mg or 100-600 mg;
c) cetirizine between 1-10 mg;
d) bilastine between 1-100 mg or 5-100 mg;
e) loratadine between 1-10 mg;
f) fexofenadine between 1-180 mg or 30-180 mg;
g) phenylephrine between 1-60 mg or 1-40 mg;
h) pseudoephedrine between 1-240 mg or 60-240 mg;
i) guaifenesin between 0-2,400 mg or 300-2,400 mg; and
j) dextromethorphan between 0-120 mg.

7. The method according to claim 3, wherein the medication is for administering in single or multiple dosage units during a 12-hour period, wherein at least one of the following ingredients is in the amounts indicated for that ingredient:
a) paracetamol between 1-2,000 mg;
b) ibuprofen between 1-600 mg;
c) cetirizine between 1-5 mg;
d) bilastine between 1-80 mg;
e) loratadine between 1-5 mg;
f) fexofenadine between 1-90 mg;
g) phenylephrine between 1-30 mg;
h) pseudoephedrine between 1-120 mg;
i) guaifenesin between 1-1,200 mg; and
j) dextromethorphan between 0-80 mg.

8. The method according to claim 3, wherein the medication is for administering at least one of the following ingredients in the amounts indicated for that ingredient:
a) paracetamol between 300-600 mg, 265-575 mg, 425-500 mg, or is 500 mg;
b) ibuprofen between 100-300 mg,125-250 mg, 125-200 mg, or is 150 mg;
c) cetirizine between 1-10 mg, 1.5-10 mg, 2-10 mg or is 2.5 mg;
d) bilastine between 1-20 mg, 1.5-20 mg, 4-20 mg, or is 5 mg;
e) loratadine between 1-10 mg, 1.5-10 mg, 2-10 mg, or is 2.5 mg;
f) fexofenadine between 1-200 mg, 20-200 mg, 40-180 mg, or is 30 mg;
g) phenylephrine between 1-20 mg, 1-10 mg or 2.5-8 mg, or is 5 mg;
h) pseudoephedrine between 5-70 mg, 15-53 mg, 20-50 mg, or is 30 mg;
i) guaifenesin between 80-380 mg, 100-300 mg, 100-200 mg, or is 150 mg; and
j) dextromethorphan between 0-25 mg, 0-20 mg, 0-10 mg, or 0-5 mg.

9. The method according to claim 1, wherein the paracetamol, the ibuprofen and the non-sedative antihistamine (or a pharmaceutically acceptable salt, or enantiomer of any mentioned aforesaid) are each for administering or taking at the same dosage event, on at least some occasions during the same day such that the doses comprise one or more of the following forms: a) tablets, b) capsule, c) aerosol and d) liquid.

10. The method according to claim 1, wherein the paracetamol, the ibuprofen and the non-sedative antihistamine (or a pharmaceutically acceptable salt, or enantiomer of any mentioned aforesaid) are each for administering or taking at different dosage events, on at least some occasions during the same day such that the doses comprise of one or more of the following forms: a) tablets, b) capsule, c) aerosol and d) liquid.
